(19) United States
(12) Patent Application Publication
(10) Pub. No.: US 2007/0059388 A1
(43) Pub. Date: Mar. 15, 2007

(54) Pharmaceutical composition containing a safe extracts of plants for the treating and preventing of diabetes and cardiovascular disease

(76) Inventor: Yaguang Liu, L. E. H., NJ (US)

Correspondence Address: Yaguang Liu, 22 Sunrise Bay Blvd., Little Egg Harbor, NJ, 08087, US

(21) Appl. No.: 11/224,641

(22) Filed: Sep. 13, 2005

(51) Int. Cl. A61K 36/899; A61K 36/42; A61K 31/715; A61K 31/704

(52) U.S. Cl. 424/750; 424/758; 514/26; 514/54

(57) ABSTRACT

This invention relates in general to the treatment of diabetes and cardiovascular disease. The pharmaceutical composition is composed of two ingredients: pumpkin polysaccharide and corn saponins and method of making the same.

### BACKGROUND OF THE INVENTION

This invention relates in general to the treatment of diabetes and cardiovascular disease. The pharmaceutical composition is composed of two ingredients: pumpkin polysaccharide and corn saponins and method of making the same.

### DESCRIPTION OF THE PRIOR ART

The major characteristic of diabetes is the body's inability to regulate the level of glucose in the blood. Therefore, the goal of treating diabetes is the reduction the blood glucose. In clinic, insulin and some oral hypoglycemic drugs, which include tolbutamide, tolazamide, acetohexamide, chlorpropamide, glyburide and glipizide, are available for treating diabetes. However, all mentioned above drugs have several disadvantages in therapeutic use. For example, some drugs increase the risk of acute cardiovascular disease. All drugs are not effective in treating the following symptoms: diabetic acidosis or in stressful situations such as infection and the degenerative diseases which cause by diabetes. More particularly, as mentioned earlier drugs are not effective in treating atherosclerosis, lose of sight, maimed and death brought about by progressive vascular injury, and in fact, above disease is main lethal reason of diabetes.

### DETAILED DESCRIPTION

Diabetes is a disease that affects at least five percent of the America population. It is the third leading cause of death in the United States. The annual incidence of diabetes is 620,000. The economic impact of diabetes is enormous, estimated at 8 billion dollars one year. Nearly 10 percent of working persons age 45 or older are diabetics.

Pumpkins are popular food commonly eaten cooked. A pumpkin is a fruit of the genus cucubita and largely cultivated in continental North America, Europe and Asia. Pumpkins are growing today in the U.S. for decoration than for food. Pumpkins are most commercial plant and it is very cheap. According to the present invention, pumpkins will be a very cheap natural source for drugs that used for prevention and treatment of diabetes.

The present invention disclosed that pharmaceutical compositions of pumpkin polysaccharide and corn saponins (PS) have a significant blood glucose reducing effect and repairing disorderly metabolism at same time and to their use as medicine, particularly in the therapy of diabetes.

Corn silk is the silky tassel inside the cornhusk. Corn silks and bracts are obtained from milling of maize, Zea mays. The national source alone could provide more than 100 million tons of corn silks and bracts in the U.S. now. A part of the corn silks and bracts can be obtained during the corn wet milling process. We are developing a new technological approach to convert abundant agricultural waste into a valuable pharmaceutical product.

Diabetes is a state of absolute or relative lack of functional insulin. It is not a single disease in the classic sense; but rather a clinical syndrome applied to a number of Pathogenetically heterogenous disorders. To be exact, Diabetes is disease characterized by abnormalities of the endocrine secretions of the pancreas resulting in disordered metabolism of carbohydrate, fat and protein, and in time, structural and functional abnormalities in a variety of tissues. It also has been established in the prior art that metabolism of carbohydrate, fat, protein and hormones, et al are regulated by liver. The liver plays a key role in regulation metabolism of carbohydrate (including glucose) and is important in many other bodily functions. It manufactures blood coagulants, stores vitamins and minerals, produces enzymes, cholesterol and proteins and neutralizes substances that would harm the body. The liver can construct the storage form of many energy sources, for example, glycogen and fats. The liver can also convert glucose to protein and fat, protein into glucose, and fat into protein or glucose. Obviously, the liver plays a key role in relation metabolism of diabetes.

For the reason given above, “composition” which can reduce blood glucose and repair disordered metabolism including increasing synthesis of RNA and protein in injured liver at same time, it is very important for treating and preventing diabetes.

In addition, the composition is very safe because all plants are pumpkin and corn. Composition can be administered to patients in the form of capsules containing a powdered mixture of the active ingredients in appropriate proportions. Alternatively, tablets can be prepared comprising the active ingredients and pharmaceutically acceptable binders, excipients, lubricants, sweeteners and coatings. A syrup or elixir may be prepared by dissolving the composition in alcohol or water together with suitable preservatives, sweeteners, dyes and flavoring agents. Ampoules or vials for injection may likewise be prepared, with the composition as prepared for oral administration being purified through further sterilization and the addition thereto of distilled water and other suitable solvents and additive known in the pharmaceutical art.

The composition dosage units prepared according to the invention can be administered to patients with a very safe and in reducing blood glucose and repairing disorderly metabolism.

The following specific examples will provide detailed illustrations of methods of producing composition according to the present invention and pharmaceutical dosage units containing composition. Moreover, examples will be given of pharmaceutical testing performed with composition that demonstrates its effectiveness in treating and preventing diabetes. These examples are not intended, however, to limit or restrict the scope of the invention in any way, and should not be construed as providing conditions, parameters, reagents, or starting materials which must be utilized exclusively in order to practice the present invention.

### EXAMPLE 1

### Extraction of Pumpkin Polysaccharide

2000 ml of 80% ethanol was added to 1000 g of dry powder of pumpkin. The mixture was distilled under reduce pressure to recover ethanol and residue obtained. The residue was further extracted with distilled water on a hot water-bath (80° C.). The hot water extract was cooled and filtered and 95% of ethanol was added to filtrate and precipitates formed. The precipitates were collected by centrifugation, washed thoroughly with ethanol and ether, and dried. A grayish white powder was obtained. The dried powder was frozen overnight and allowed to thaw at room temperature. By the repeated freezing and thawing procedure, the powder was extracted by a cold water and removed soluble components. The resulting residue was chromatographed on DEAE-cellulose column. The column was eluted with hot water. The elution was concentrated by evaporation under reduced pressure. The residue obtained and residue was freeze-dried. The final product is pumpkin polysaccharide.

### EXAMPLE 2

### Corn Saponins

One kg dried powder of corn silks or bracts were extracted with 2000 ml of 95% ethanol at room temperature for 24 hours. The powder was recovered by filtration. Filtrate A was saved and the powder filtercake was refluxed with an additional 2000 ml of 95% ethanol on a steam bath. The mixture was filtered again. Filtrate B was saved and the powder filtercake was refluxed two more times for 6 hours with additional 2000 ml batches of 95% ethanol and filtered, provided filtrates C and D. Filtrates A, B, C, and D were combined and distilled under reduced pressure, whereby ethanol was recovered and a still residue was obtained.

This still residue was dissolved in 500 ml of distilled water. This water solution was extracted five times with 500 ml of a lipophilic solvent, e.g. diethyl ether or petroleum ether, whereby lipids were removed from the solution.

To this aqueous raffinate was added 500 ml of water-saturated n-butanol and the mixture was distilled under reduced pressure to dryness, whereby a powder residue was obtained. This powder was dissolved in 500 ml of anhydrous ethanol, and 2000 ml of acetone were added with agitation while a precipitate forms. The precipitate was recovered by filtration and washed twice with acetone and twice with diethyl ether and dried. About 60 g of a white of light yellow powder were recovered. This is corn saponins.

### EXAMPLE 3

### PS Injecting Preparation

PS, according to the conventional methods, was made as ampoules or other injection preparation, and then sterilized. Type XGI.S double door functional ampoule sterilizing machine is used for manufacturing of PS injection. The function of facilities includes sterilization, leakage detection and washing. Microcomputer (PC machine) is applied in the principal controlling system. Dose is intramuscularly 5-100 mg daily.

### EXAMPLE 4

### PS Oral Preparation

PS powder granulated accorded to the conventional granulation method. The mixture content decreased from 100% to 93%. The 7% of content was different types of fillers. Disintegrants and lubricants were used: microcrystalline cellulose, microfine cellulose, lactose cellulose granulate, α-lactose monohydrate, and modified maize starch.

The disintegrants tested were the following: cross-linked sodium carboxymethyl cellulose, Cross-linked calcium carboxymethyl-cellulose, potato starch, sodium starch glycolate, cross-linked polyvinylpyrrolidone, and low-substituted hydroxypropyl-cellulose.

For lubrication, the following were used: magnesium stearate, glyceryl tristearate, and polyethylene glycol.

Colloidal silicon dioxide and hydrophobic colloidal silicon dioxide was added. The content of PS was kept constant at a level of 100 mg per tablet. Tablet weight was varied between 100-105 mg. Tablet mixtures were mixed for 10 min in the Turbula mixer. The lubricants were sieved through a 315-μm sieve into the mix. Final mixing was carried out for 5 min at 42 rpm in the Turbula mixer. The mixtures were compressed using a rotary press. The lower compression roller was instrumented with four strain gauges. A Philips carrier-frequency bridge was used for signal amplification. Each batch was compressed at different levels of compression. As a stabilizer, ascorbic acid was added. Sugarcoating operation was also performed conventionally.

The dosage of PS is orally 50-200 mg daily.

### EXAMPLE 5A

### Preparation of Crude Composition

Crude composition is extracted from as mentioned above plants by ethanol and water. Proportion of plants, for example, is as following (by weight):

Weight Percent

| | |
|---|---|
| Pumpkin | 20 to 50% |
| Corn silks or bracts | 20 to 70% |

The tissues of plants were dried and powdered. 5 liters distilled water was added 1 kg of dried powder. The solution was heated to boil and simmered for one hour after boiling. This water extraction was repeated two times. Combined and filtered. The filtrate was concentrated under reduced pressure to approximately 500 ml. Then 2,000 ml of 90% ethanol was added to 500 ml water solution. Stir. Stilled. Filtered. Residue and filtrate (1) was obtained. 1,000 ml 90% ethanol was added to residue. Stir. Stilled. Filtered. Filtrate (2) was obtained.

Combined filtrate (1) with (2). Then total filtrate was concentrated to syrup under reduced pressure distillation. Ethanol was recovered. Syrup dried under vacuum drying. Granulated to final powder. Weight of every capsule and table is about 200-500 mg. Crude-composition is similar to fine-composition in pharmacological property. The following examples are related to pharmacological tests.

### EXAMPLE 5B

### Preparation of Fine Composition

Fine composition according to the present invention consists of:

Weight Percent

| | |
|---|---|
| Pumpkin polysaccharide | 30 to 70% |
| Corn saponins | 30 to 70% |

The dry ingredients or derivate of ingredients prepared in accordance with the present invention, may be incorporated tablets, capsules, syrups or other form by conventional method.

### EXAMPLE 6

### Hypoglycemic effect of composition

Experiments use alloxan diabetic mice. Male mice 18-22 g was used in these experiments. The diabetic mice had high blood glucose, produced by a single dose of alloxan 75 mg/kg intravenously. Inject 2 ml of normal saline into the peritoneal cavity of mouse for control and 100 mg/kg composition group daily. Blood samples were collected from ocular venous plexus of mice.

The blood glucose levels were determined according to hexokinase method. The procedure is as the following:

A. Reagents

1. Vial B, containing NADP. Reconstitute by adding 15.5 ml water and gently swirling.

2. Vial A. Add the entire contents of vial B to vial A and dissolve by gently inversion.

According to the manufacturer, the reagent has the following composition:

a. Tris buffer, pH 7.5, 50 mmol/L

b. ATP, 0.5 mmol/L

c. NADP⁺, 0.45 mmol/L

d. Mg⁺⁺, 17 mmol/L

e. Hexokinase, 666 U/L

f. G6PD, 333 U/L

3. Stock Standard Glucose, 10.0 g/L. Dissolve 1.0 g pure anhydrous D-glucose in water containing 1.0 g benzoic acid per liter. Make up to 100 ml volume in the benzoic acid solution.

4. Working Glucose Standards. Prepare standards of 50, 100, 200, and 400 mg/dl by appropriate dilution of Stock Standard with benzoic acid solution.

B. Procedure

1. Place 1.5 ml prepared reagent in a series of cuvets for standard, unknowns, and control serum, respectively.

2. Appropriate blanks are set up by placing 1.5 ml of 9 g/L NaCl in a series of cuvets.

3. After incubating for 5 or 10 minutes, read the absorbance of each cuvet at 340 nm and check again a few minutes later to insure that an end point has been reached.

TABLE 1
Blood glucose level (mg/dl)

| C | T |
|---|---|
| 251 ± 50 | 160 ± 18.8 |

Number of samples: 20;
P < 0.01;
C: Control group;
T: Treatment group

From above results, it is apparent that composition can obviously decrease blood glucose levels.

### EXAMPLE 7

### Effect of Composition on Binding Insulin Receptor

Rats were sacrificed by a blow on the head, and their epididymal adipose tissue were quickly removed. The fat cells were isolated from the adipose by the procedure of Rodbell (Rodbell, M.: J. Biol Chem, 239:375, 1964). In dulbecco buffer PH 7.4 containing collagenase (3 mg/ml) and albumin (40 mg/ml).

¹²⁵I-labeled insulin (¹²⁵I-insulin) was at specific activities of 100-200 μCi/g. IgG was prepared from heparinized plasma. The IgG fraction of serum from the patient with the highest concentration of antireceptor antibody activity (B-2) was prepared from the ammonium sulfate precipitate by ion exchange chromatography of DEAE-cellulose.

Antireceptor antibodies were assayed by methods of inhibition of ¹²⁵ I-insulin binding to cultured human lymphoblostoid cells. The cells were prepared: 2-4.times.10.sup.6 cells/ml of adipocytes cells were washed three times for 10 minutes at 37° C. and nondissociable radioactivity was extracted in 1% triton X-100. ¹²⁵ I-insulin binding to isolated rat adiposytes were performed at 37° C. in krebs-ringer bicarbonate medium (PH 7.4) containing bovine serum albumin and bacitracin (100 Upper milliliter). After adipocytes had been incubated with ¹²⁵ I-insulin for 30 minutes at 37° C., the cells were precipitated from the medium by centrifugation. The radioactivity in the pellet was counted.

TABLE 2
The binding of insulin receptor

| C | T |
|---|---|
| 100% | 125 ± 15 |

Number of samples: 20;
P < 0.05

From above results, it is apparent that composition can obviously stimulate binding insulin receptor with insulin.

### EXAMPLE 8

### Effect of Composition on Synthesis of Protein

The 20-22 g male mice were used in experiments. The mice were injected with CCl.sub.4. The dosage of composition was 75 mg/kg injected intraperitoneally. The control mice were injected with same volume of normal saline. The mice were sacrificed by decapitation. Their liver was quickly excised and placed immediately in cold Medium which consisting of 0.25M sucrose, 0.065M potassium chloride, 0.035M potassium bicarbonate, 0.01M magnesium chloride and 0.05M tris (hydroxymethyl)aminomethane (Tris), adjusted to pH to 7.5 with HCl. The liver was cleaned of excess fat before the wet weight was measured. The liver from each animal was homogenized in each experiment. All operations were performed at 4.degree. C. Each liver was homogenized in 10 ml of cold Medium, Using Teflon and glass homogenizer immersed in ice. The homogenate was centrifuged at 1000 g for 10 minutes to remove large cellular particles. The resulting supernatant fluid was filtered through four layers of cloth to remove as much fatty material as possible. The filtrate was centrifuged at 37,000 g for 30 minutes. The sediment was discarded, and the resulting postmitochondrial fraction was used for the assay of translation. Protein concentration was measured by the biuret procedure [J. Biol Chem 177:751, 1949], using crystalline bovine serum albumin as a standard. The rate of translation was determined in an assay system containing: 0.2 ml of 0.01M ATP, 0.2 ml of 0.05M phospho puruvate, 0.05 ml of a ³H-amino acid mixture (containing approximately 5×10⁶ cpm), 0.05 ml of crystalline pysuvate kinase (1 mg/ml), 0.1 ml of water and 1.0 ml of the postmitochondrial preparation in Medium in a total volume of 2 ml. The postmitochondrial preparation was added last to initiate the reaction, and the mixture was incubated for 30 minutes at 37° C. Under the conditions of the experiment, translation was a straight-line function of time for at least 45 minutes. The course of the reaction was halted by the addition of 5 ml of 10% trichloroacetic acid (TCA). Control tubes were prepared by adding all of the components of the reaction mixture into 5 ml of 10% TCA. The precipitated proteins were collected on a 0.45-μm membrane filter, using vacuum filtration. The collected precipitate was washed two times with 20 ml portions of 10% TCA and dried in an oven at 80° C. for 10 minutes. The dried filters were placed in scintillation vials containing 20 ml of Aquasol, and the radioactivity that had been incorporated into protein was measured in a liquid scintillation counter.

TABLE 3
CPM/mg proteins

| C | T |
|---|---|
| 560 ± 75 | 860 ± 76 |

Number of samples: 20;
P < 0.01

The data of Table 3 indicated that PS could increase protein synthesis of liver.

### EXAMPLE 9

### The Effects of Composition on Ribonucleic Acid (RNA)

The method of animal is like procedure of example 8. ³H-uridine (10 μCi/100 g body weight) was injected intraperitoneally into mice 20 minutes prior to sacrifice. Their liver was quickly excised. Livers were washed with cold 0.25M sucrose containing 3.3 mM CaCl₂ and minced with scissors. The mince was then homogenized with 3 volumes of the same solution in a Potter's homogenizer with a glass pestle and centrifuged at 1000.times.g for 10 minutes. The sediment was homogenized with 3 volumes of 0.25M sucrose-3.3 mM CaCl.sub.2 in a Potter's homogenizer with a Teflon pestle. The homogenates were filtered through 4 layers of gauze. Eight volumes of 2.2M sucrose was added and the mixture was centrifuged at 40000.times.g for 1 hour to sediment the nuclei. Purified nuclei were washed with 0.6N perchloric acid, ethanol and ether. To the residues was added 0.5N KOH and the mixture was incubated at 37° C. for 18 hours, followed by acidification to remove deoxyribonucleic acid (DNA) and proteins as precipitates. After centrifugation the supernatant was neutralized with KOH. Radioactivity incorporated into nuclear RNA and the amount of RNA was determined using aliquotes of this supernatant. Radioactivity was counted in a scintillation spectrometer with solution, the composition of which was as follows: one liter of the solution contained 50 ml of methanol, 10 ml of ethyleneglycol, 60 g of naphthalene, 4 g of 2,5-diphenyloxazole, 0.2 g of 1,4-bis [2(5phenyloxaxolyl)]-benzene and dioxane.

TABLE 4
Specific radioactivity (CPM/mg RNA)

| C | T |
|---|---|
| 18090 ± 1819 | 30200 ± 2500 |

Number of samples: 20;
p < 0.01;

The data of Table 4 indicated that PS could obviously increase RNA synthesis.

### EXAMPLE 10

### Effect of PS on Apoptosis of Smooth Muscle Cells of Atherosclerotic Rabbits

Methods

Smooth muscle cells of atherosclerotic rabbits were grown in RPMI Medium 1640 supplemented with 10% (v/v) heat-inactivated FBS (56° C. for 30 min) at 37° C. in a humidified 95% air/5% CO₂ atmosphere. Cells were seeded at a level of 2×10⁵ cells/ml. Cells were allowed to attain a maximum density of 1.2×10⁶ cells/ml before being passed by dilution into fresh medium to a concentration of 2×10⁵ cells/ml.

Apoptosis determined by two methods:

Method (1): Cell pellets containing 5×10⁶ cells were fixed with 2.5% glutaraldehyde in cacodylate buffer (pH 7.4), dehydrated through graded alcohol, and infiltrated with LX-112 epoxy resin. After overnight polymerization at 60° C. 1-μm sections were cut with glass knives using a LKB Nova microtome. The sections were stained with 1% toluidine blue and coverslipped. In addition, experimental examples were stained with May-Grunwald-giemsa stain for the demonstration of apoptosis.

DNA electrophoresis: Untreated and treated cells collected by centrifugation, washed in phosphate buffered saline and re-suspended at a concentration of 5×10⁶ cells and 0.1% RNase A. The mixture was incubated at 37° C. for 30 min and then incubated for an additional 30 min at 37° C. with 1 ml protease K. Buffer was added and 25 μl of the tube content transferred to the Horizontal 1.5% agarose gel electrophoresis was performed at 2 V/cm. DNA in gels visualized under UV light after staining with ethidium Bromide (5 μg/ml).

DNA fragmentation assays: DNA cleavage was performed, quantitation of fractional solubilized DNA by diphenylamine assay and the percentage of cells harboring fragmented DNA determined by in labeling techniques. For the diphenylamine assay, 5×10⁶ cells were lysed in 0.5 mL lysis buffer (5 mmol/L Tris-HCl, 20 mmol/L DTA, and 0.5% Triton X-100, pH 8.0) at 4° C. Lysates were centrifuged (15,000 g) for separation of high molecular weight DNA (pellet) and DNA cleavage products (supernatant). DNA was precipitated with 0.5 N perchloric acid and quantitated using diphenylamine reagent. The cell cycle distribution was determined 4 hours after addition of drug and represents mean±SD of 5 independent experiments.

Method (2): Apoptosis of smooth muscle cells was assessed of atherosclerotic rabbits by changes in cell morphology and by measurement of DNA nicks using the Apop Tag Kkt (Oncor, Gaithersburg, Md.). Morphologically, HL-60 cells undergoing apoptosis possess many prominent features, such as intensely staining, highly condensed, and/or fragmented nuclear chromatin, a general decrease in overall cell size, and cellular fragmentation into apoptotic bodies. These features make apoptotic cells relatively easy to distinguish from necrotic cells. These changes are detected on cytospin preparations stained with Diff-Quick Stain Set. Apoptotic cells were enumerated in a total of about 300 cells by light microscopy.

For evaluation of apoptosis by flow cytometry, cells were fixed and permeabilized in 1% paraformaldehyde and ice-cold 70% ethanol. Digoxigenin-dUTP was incorporated at the 3′OH ends of the fragmented DNA in the presence of terminal deoxynucleotidyltranserase, and the cells were incubated with FITC-labeled anti-digoxigenin-dUTP and with propidium iodide. Green (apoptotic cells) and orange (total DNA) fluorescence were measured with a FACScan flow cytometer and analyzed with LYSIS II and CELLFIT programs.

TABLE 5
Effect of PS on apoptosis of smooth muscle cells

| Drug | Apoptosis (%) |
|---|---|
| Control | 10.8 ± 1.8 |
| PS | 3.2 ± 0.5* |

*P < 0.001 compared with control group.

The data of Table 5 indicated that PS could significantly decrease apoptosis of smooth muscle of atherosclerotic rabbits. It means PS can decrease the development of atherosclerotic disease.

### EXAMPLE 11

### The Influence of PS on Aggregation of Platelets

Methods for Blood of Humans:

Blood was collected from veins of humans using a needle attached to a plastic disposal syringe. The blood was immediately transferred into siliconized glass tube containing 0.1 volume of 3.13% sodium citrate. Platelet-rich plasma (PRP) was obtained by centrifugation of the whole blood at 1000 rpm for 10 min at room temperature. Platelet-poor plasma (PPP) was prepared by centrifugation of the remaining blood at 3000 rpm for 10 min. Platelet aggregation was performed using in aggregameter at 37° C. Human platelet studies were carried out at constant platelet number (3×10⁸/ml). With regards to determination of platelet aggregation, the maximum aggregation induced by adenosine diphosphate (ADP) in a final concentration of 2 μM was obtained by the light transmission method. 0.4 ml PRP of each subject was introduced into each of 24 tubes and divided into 2 groups. Then to the 12 tubes of each group were added 50 μl of saline and 50 μl PHP (0.5 mg/ml) respectively. After incubation of 3 min at 37° C., to each of 12 tubes of each group were added 50 μl of 2 μM ADP. A 5 minutes aggregation curve for each tube was plotted.

Statistical analysis of the results was carried out using Student's t-test for paired data.

TABLE 6
Aggregation rate of platelet (%)

| C | T |
|---|---|
| 50.4 ± 8.1 | 18 ± 2.1 |

Number of samples: 20;
P < 0.001

### EXAMPLE 12

### The Influence of PS on Lowering Heperlipidemia

As test animals, 30 male mice weighing of 12-22 grams (g) was used. They were divided into the following three groups. Each group consists of ten mice.

(A) Standard Group

Each animal in standard group is given a daily 0.5 ml of distillatory water by stomach-tube.

(B) Control Group

Each animal in control group is given a daily 0.5 ml of cholesterol-emulsion. Cholesterol-Emulsion has the following materials:

a. cholesterol: 5 g

b. sodium deoxycholate: 0.5 g

c. lard: 10 g

d. tween: 10 ml

e. propylene glycol: 10 ml

Water was added: 50 ml.

(C) PS Group

Each animal in PS group is given a daily 0.5 ml of cholesterol-emulsion and about 80 mg/kg of PS. Mice of three groups were nurtured using a standard diet.

The mice of the above three groups were raised for 10 days. At the end of 10-day test period the animals are weighed and sacrificed, serum cholesterol and triglycerides of liver are determined from blood. The methods are described as following: Chemical colorimetric methods have been used in the analysis of serum cholesterol.

(D) Reagents and Materials

1. Isopropanol, reagent grade.

2. Adsorbent Mixture for the removal of bilirubin, phospholipids, monoglycerides, diglycerides, glucose, and other chromogenic material. Mix well the following materials:

a. Alumina 50-100 mesh, 900 g.

b. Zeolite (Taylor), group and sifted to 20-80 mesh, activated by heating at 110° C. overnight, 50 g.

c. Lloyd's reagent, 50 g.

d. CuSO₄ anhydrous powder, 10 g.

e. Ca(OH)₂ anhydrous, 20 g.

3. Sulfuric Acid, concentrated, reagent grade.

4. Ferric Chloride Color Reagent. Place 500 mg FeCl₃ 6H₂ 0 in 500 ml volumetric flask, add glacial acetic acid to the mark, and mix. The reagent is stable in the dark for 1 year at room temperature.

5. Cholesterol Standard, 200 mg/dl. Dissolve 200.0 mg cholesterol in isopropanol and make up to 100 ml volume.

6. 20×150 mm screw-capped culture tubes with teflon-lined caps.

(E) Procedure

The development and measurement of the color after preparation of the extract has been made by the following methods:

1. Extraction: Pipet 9.5 ml isopropanol into all culture tubes to be used for samples and controls and 9.0 ml isopropanol plus 0.5 ml water to tubes for standards.

2. Pipet 0.5 ml of serum into the appropriate sample and control tubes and 0.50 ml of cholesterol standard into the standard tubes. Tightly stopper and shake vigorously or mix on a vortex-type mixer for 20 seconds.

3. Allow to standing about 20 minutes, adding about 2 g of adsorbent mixture to each tube, and mix thoroughly for 20 seconds. Let stand for 30 minutes, and shake vigorously for 5 second every 10 minutes.

4. Centrifuge for 10 minutes at 1100 to 1200 g. Aliquots of the extract can be used for the determination of cholesterol. Samples with grossly elevated concentrations can be diluted with isopropanol and re-assayed.

5. Color reaction. Prepare a blank by pipetting 1.0 ml of isopropanol into a tube. Transfer 1.0 ml of sample, control, and standard extracts, respectively, into appropriately marked tubes.

6. To each tube, add 2 ml FeCl₃ reagent and mix.

7. Add 2 ml concentrated H₂SO₄ to a tube by allowing the acid to run down the side of the slanted tube, tightly stopper, and mix by inversion 6 times. Then proceed to the next tube.

8. Let color develop for 10 minutes, transfer to a cuvet, and read the absorbance against the blank at 540 nm.

9. Calculations. Let Aᵤ be absorbance of sample and A_{S} the absorbance of standard; read against the blank.

TABLE 7

| | Cholesterol of serum | Triglyceride of liver |
|---|---|---|
| | (mg/dl) | (mg/100 g) |
| Control group | 420 ± 20 | 27.9 ± 4.5 |
| PS group | 275 ± 30 | 18.3 ± 2.5 |

P < 0.01;
Number of samples: 20

The data of examples 10-12 indicated that PS could treat and prevent cardiovascular disease.

### EXAMPLE 13

### Safety of PS (1): Toxic Dose for Mice

Methods for Determination of LD₅₀

Mice were used in the experiment. The animals were assigned by weight into the treatment and control groups. The animals were singly housed in hard-bottomed polypropylene cages with wood shavings. The animals had free access to food and water. Lighting was controlled on a 12 hours light; 12 hours dark cycle, (lights on 8 a.m.; lights off 8 p.m.). The housing facility temperature was maintained at 20°±2°. Humidity was maintained between 50-70%.

Parameters Assessed

Bodyweight, food and water intake. Prior to commencement of the study, all animals were weighed and assigned to groups, ensuring all groups had a similar mean weight. The body-weight of each animal was recorded prior to drug administration, as was food and water. These values were recorded again 24 hours later and the body-weight change, food and water intake was calculated as the difference between these three measurements.

Home cage activity. Animals were singly housed in a home cage monitor and their activity monitored during the nocturnal period (8 p.m.-8 a.m.), throughout the study. The cage in which the animal is housed (home cage) is placed into a compartment on a rack. On the top of each compartment there is a passive infra-red (PIR) sensor. The sensor is powered by a 10 volt direct current power supply. This splits the infra-red beam into 16 zones which radiate across the floor of the cage. The 24 sensors are connected by separate switch inputs to an interpak 2 interface. The whole system is controlled by the home cage activity monitor software package. The data are listed as below.
LD₅₀: The LD₅₀ of PS in mice (I.P.) was found to be 2.5 g/kg.
Toxic doses for mice: In 38 normal mice after injection of PS of 2.5 g/kg/day×5 with the observation period of 5 days none of the mice died.
   As to subacute toxicity tests, a dosage corresponding to 50 times the clinical dose is administered continually for two months, and no side effects have been observed. The electrocardiograms and functions of liver and the kidney have not been affected and no injuries whatever have been observed in the tissue slices of the heart, liver, spleen, lungs, kidneys and adrenal.

### EXAMPLE 14

### Safety of PS (2): Analysis of Chromosomes

For metaphase chromosomes, kidney cell cultures were treated with colchicines (0.4 μg/ml) for 3-4 hours. The cells were then trypsinized and treated with hypotonic solution (0.075 M KCl) at 37° C. for 30 minutes. The cell suspensions were centrifuged and the pellets fixed in cold acetic acid:methanol (1:3) solution. Slides were prepared by standard air-drying method and stained with 2% Giemsa solution. The results scored by analyzing at least 200 well spread metaphases with 44±2 chromosomes for gaps, chromatid and chromosome breaks and exchanges, and association. Chromatid and chromosome aberrations were scored separately, and the total percentage was subjected to statistical analysis. Gaps were recorded but not included in the total frequency. Endoreduplication (endomitosis) was estimated from at least 500 cells/animal and expressed as a percentage.

TABLE 8
Chromosomal aberrations induced by PS in kidney

| | Aberration/100 cells | | |
|---|---|---|---|
| Duration of treatment | Chromosome | | % Aberrant cells |
| (months) | Breaks | Exchanges | (mean ± SEM) |
| Untreated | | | |
| 0.5 | nd | nd | 0.2 ± 0.1 |
| 1.0 | nd | nd | 0.3 ± 0.2 |
| 2.0 | nd | nd | 0.3 ± 0.1 |
| 3.0 | 0.1 | nd | .0.5 ± 0.3 |
| 4.0 | nd | 0.1 | 0.5 ± 0.2 |
| 5.0 | 0.1 | nd | 0.4 ± 0.2 |
| PS | | | |
| 0.5 | 0.1 | nd | 0.2 ± 0.08 |
| 1.0 | 0.5 | nd | 0.2 ± 0.10 |
| 2.0 | 0.7 | 0.1 | 0.3 ± 0.15 |
| 3.0 | 0.9 | nd | 0.3 ± 0.16 |
| 4.0 | 1.2 | 0.1 | 0.3 ± 0.20 |
| 5.0 | 1.3 | 0.2 | 0.3 ± 0.20 |

The data of Table 8 indicated that PS has no exchange in chromosome, no chromatid or chromosome aberrations and no significant differences in the frequency of either chromosome lesions or chromatid or chromosome aberrations with increasing age.

### EXAMPLE 15

### Safety of PS (3): Mutagenic Effect of PS

Determination of the mutagenic and carcinogenic activity is important for estimating side effects of drug. The mutagenic activity of many drugs can only be detected with growing cells. In present study, mutagenic and carcinogenic activity of PS is determined by Bacteria system.

The method for detecting mutagenicity of PS, with the Salmonella system that detects the reversion of the bacteria from His⁻ to His⁺, is widely used. Methods for detecting carcinogens and mutagens with the salmonellia mutagenicity test are highly efficient in detecting carcinogens and mutagens. Major carcinogens tested have been detected as mutagens. Salmonella mutagenicity assay is very sensitive and simply test for detecting mutagens and carcinogens. Therefore, it has been useful in a detailed study that has been made of mutagenic activity of PS.

TAa7, TAa8, TA100 and TA102 are extremely effective in detecting classes of carcinogens and mutagenesis.

Methods

The bacterial tester strains used for mutagenesis testing are TA97, TA98, TA100 and TA102. Mutagenesis testing method was done as described previously. In brief, TA97, TA98, TA100 and TA102 were grown in agar gel culture. The petri plats (100×15 mm style) contain 30 ml with 2% glucose. The agar mixture was agitated vigorously and immediately poured into plates of minimal agar. The cultures were incubated at 37° C. in a dark and 5% CO₂ in air for 48 hours. After 48 hours the colonies in both test and controls are counted. The presence of a background lawn of bacteria on the histidine-poor soft agar plate was used as an indication that gross toxic effects were absent. Mutagenicity assays were carried out at least in triplicate.

Results and Discussion

The data of experiment summarized as the following table.

TABLE 9
Mutagenesis Assay on plates

| | Dose/ | Number of His⁺ revertants/plate | | | | | |
|---|---|---|---|---|---|---|---|
| | plate | TA97 | | TA98 | | TA100 | |
| Treatment | (μg) | −S | +S | −S | +S | −S | +S |
| Spontaneous | — | 149 ± 15 | 150 ± 17 | 35 ± 4 | 36 ± 4 | 120 ± 17 | 120 ± 15 |
| 4NQO | 0.5 | 861 ± 79 | — | 338 ± 35 | — | 2301 ± 190 | — |
| PS | 1000 | 150 ± 16 | 150 ± 17 | 34 ± 4 | 34 ± 4 | 128 ± 15 | 130 ± 15 |

*4QO: 4-nitroquinoline-1-Oxide

The salmonella typhimurium strains TA97, TA98 and TA100 were checked using 4-nitroquinoline-1-oxide. The range of spontaneous mutation rates for the individual strains, which were considered to be acceptable, was TA97 (100-170), TA98 (20-40) and TA100 (80-150).

The data of Table 9 indicated that the number of His+ revertants/plate of PS almost is as same as spontaneous of testing strains. On the contrary, 4NQO is mutagenic and carcinogenic agent. The number of His+ revertants/plate of 4NQO is higher than 10 times of spontaneous.

In conclusion, PS has no carcinogenic and mutagenic action.

The preparation of composition is simple and can be accomplished by the extraction methods set forth above or any conventional methods for extracting the active ingredients. The novelty of the present invention resides in the mixture of the active ingredients in the specified proportions at invention and in the preparation of dosage units in pharmaceutically acceptable dosage form. The term “pharmaceutically acceptable dosage form” as used hereinabove includes any suitable vehicle for the administration of medications known in the pharmaceutical art, including, by way of example, tablets, capsules, syrups, elixirs, and solutions for parenteral injection with specified ranges of composition.

It will thus be shown that there are provided compositions and methods which achieve the various objects of the invention, and which are well adapted to meet the conditions of practical use.

As various possible embodiments might be made of the above invention, and as various changes might be made in the embodiments set forth above, it is to be understood that all matters herein described are to be interpreted as illustrative and not in a limiting sense.

1-4. (canceled)

5. A natural drug, according to claim 3, wherein said the PS has no exchange in chromosome.

6. A natural drug, according to claim 3, wherein said the PS has no chromatid or chromosome aberrations.

7. A natural drug, according to claim 3, wherein said the PS has no significant differences in the frequency of either chromosome lesions or chromatid or chromosome aberrations with increasing age.

8. A natural drug, according to claim 3, wherein said PS has no carcinogenic and mutagenic action.
